(19) United States
(12) Patent Application Publication
(10) Pub. No.: US 2005/0101006 A1
(43) Pub. Date: May 12, 2005

(54) Preparation and surface modification of plastic microfluidic chip

(76) Inventors: Ji-Yen Cheng, Taipei (TW); Cheng-Wey Wei, Taipei (TW)

Correspondence Address: Pavel POGODIN, Esq., 617 North Delaware Street, San Mateo, CA, 94401, US

(21) Appl. No.: 10/705,698

(22) Filed: Nov. 8, 2003

(51) Int. Cl. C12M 1/34; B05D 3/00

(52) U.S. Cl. 435/287.1; 427/2.11

(57) ABSTRACT

The present invention discloses a method for preparation and surface modification of plastic microfluidic chip is disclosed. A microfluidic chip is first prepared by micromachining using a laser scriber on a plastic substrate, such as a PMMA substrate, to have a desired trench width or trench widths and desired aspect ratio. Surface modification is obtained after a thermal annealing treatment, followed by a chemical modification process. During the chemical modification diverse functional groups such as perfluoroalkyl (—CₙF₂ₙ₊₂), amino (—NH₂) or sulfhydryl (—SH) are introduced to perform surface passivation or further biomolecules immobilization. A plastic microfluidic chip with modified surface characters is thus obtained. The present invention also discloses plastic microfluidic chips so prepared.

### FIELD OF THE INVENTION

The present invention relates to preparation and surface modification of plastic microfluidic chip, especially to direct write laser micromachining on plastic substrate and chemical surface modification of microfluidic chip so prepared.

### BACKGROUND OF THE INVENTION

The use of direct write laser micromachining on plastic substrates to fabricate microfluidic chip has the potential for fast prototyping and mass production. The direct write laser micromachining is fast and flexible. Most laser scribers are compatible to popular computer-aided design tools. Designers of microfluidic chips are allowed to realize their design and tests on such chips are easily conducted. Direct write laser may be used in mass production of microfluidic chips with versatility. However, the advantage of direct write laser micromachining on plastic substrate has been diminished by the rugged surface of the microchannel so prepared. Chemical surface modification of the microfluidic channel has been proposed, with limited effects.

Klank et al. disclosed a laser machining method using swivel mirror for 100×100 mm PMMA (poly (methyl methacryolate)) substrate to produce a microfluidic chip. See H. Klank et al.: “CO₂-laser micromachining and back-end processing for rapid production of PMMA-based microfluidic systems”, Lab on a chip 2 (2002), pp. 242-246. However, the problem of rough surface after laser machining persists.

Henry et al. disclosed a method of surface modification of PMMA microfluidic chip using lithodiamines. See Henry et al.: “Surface modification of poly(methyl methacrylate) used in the fabrication of microanalytical devices”, Anal. Chem., 72 (2000), pp. 33-37. Karandikar et al. disclosed another method of surface modification of PMMA microfluidic chip using ethylene diamine. See Karandikar et al.: “Homogeneous and heterogeneous modification of poly(methyl methacrylate) with ethylene diamine”, Polymer Prep., 30 (1989), pp. 250-251. These approaches, however, required extensive solvent drying procedures. In addition, the above approaches can only provide surface with amino (—NH₂) functional groups.

### OBJECTIVES OF THE INVENTION

It is thus an objective of this invention to provide a method for preparation of plastic microfluidic chip with modified surface character.

It is also the objective of this invention to provide a method for surface modification of smoothness of plastic microfluidic channel.

It is also the objective of this invention to provide a simplified method for surface modification of plastic microfluidic channel.

It is also the objective of this invention to provide a method for preparation of plastic microfluidic chip with enhanced immobilization of biomolecules.

It is also the objective of this invention to provide a method for universal chemical modification of plastic microfluidic chip.

It is also the objective of this invention to provide a plastic microfluidic chip prepared according to the above methods.

### SUMMARY OF THE INVENTION

According to this invention, a method for preparation and surface modification of plastic microfluidic chip is disclosed. A microfluidic chip is first prepared by micromachining using a laser scriber on a plastic substrate, such as a PMMA substrate, to have a desired trench width or trench widths and desired aspect ratio. Surface modification is obtained after a thermal annealing treatment, followed by a chemical modification process. During the chemical modification diverse functional groups such as perfluoroalkyl (—CₙF₂ₙ₊₂), amino (—NH₂) or sulfhydryl (—SH) are introduced to perform surface passivation or further biomolecules immobilization. A plastic microfluidic chip with modified surface characters is thus obtained.

The above objectives and advantages of this invention may be clearly understood from the detailed description by referring to the following drawings.

### BRIEF DESCRIPTION OF THE DRAWINGS

FIG. 1 illustrates the flowchart of the method for preparation and surface modification of plastic microfluidic chip.

FIG. 2 shows the cross-sectional view of trenches in cast PMMA carved with various later power and passes, after being thermally annealed.

FIG. 3 shows the SEM close-up of a sealed channel carved with 1.5 W laser power and at speed of 36 mm/sec., after being thermally annealed.

FIG. 4a shows an SEM picture of the rugged interior surface of the trench after laser machining and FIG. 4b shows the SEM picture of the same surface after thermal annealing.

FIG. 5a shows the ATR-FTIR spectra of the pristine PMMA surface and FIG. 5b shows the ATR-FTIR spectra of the LAH treated PMMA-surface.

FIGS. 6a-d show the XPS spectra of various organosilane modified surfaces according to the method of this invention.

### DETAILED DESCRIPTION OF THE INVENTION

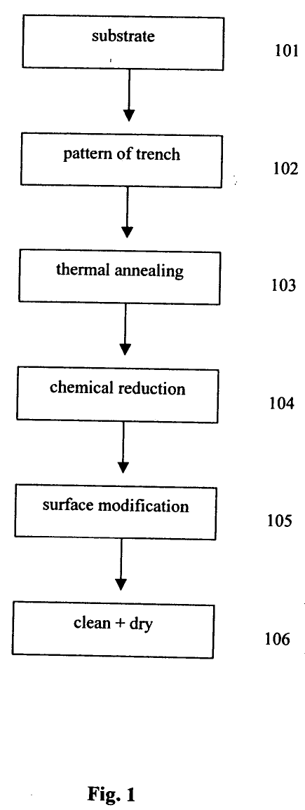

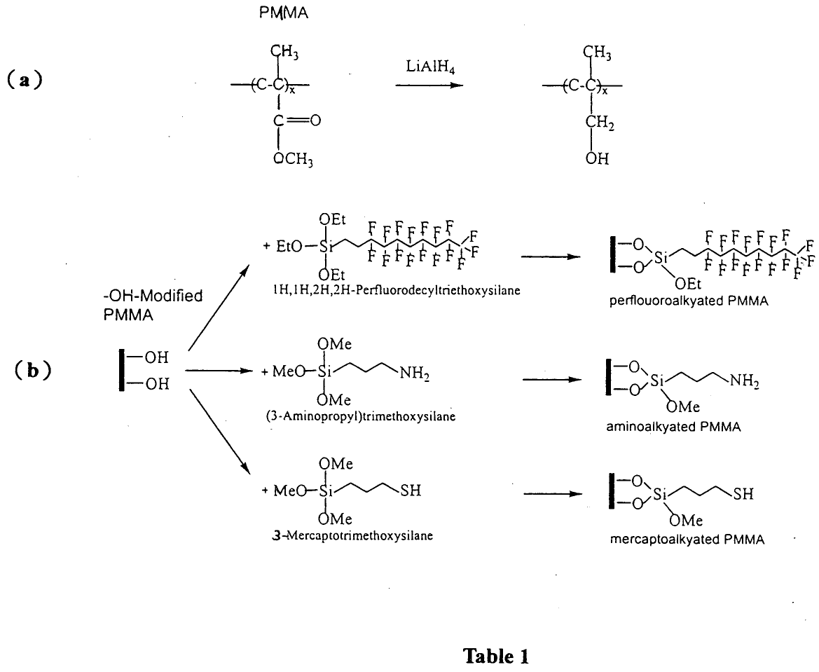

The present invention disclosed a method for preparation and surface modification of plastic microfluidic chip is disclosed. FIG. 1 shows the flowchart of the method for preparation and surface modification of plastic microfluidic chip of this invention. As shown in this figure, at 101, a plastic substrate is prepared. The plastic substrate is preferably a PMMA substrate. At 102 the plastic substrate is micromachined using a laser scriber to form a pattern of trench in the plastic substrate. Desired trench width or trench widths and desired aspect ratio of the microfluidic channel are thus obtained. At 103 the substrate is subjected to physical surface modification using thermal annealing treatment. At 104 the surface of the substrate is treated with chemical reduction to reduce the ester group on the plastic surface to produce hydroxyl groups (—OH). Suited reduction agent includes lithium aluminum hydride (LAH). At 105 the treated substrate is treated with a surface modification agent. Suited surface modification agent includes those chemicals with functional groups such as perfluoroalkyl (—CₙF₂ₙ₊₂), amino (—NH₂) and sulfhydryl (—SH). In the embodiments of this invention, surface modification agents used are 1H, 1H, 2H, 2H-Perfluorodecyltriethoxysilane, 3-(Aminopropyl)trimethoxysilane and 3-Mercaptotrimethoxysilane. At 106 the substrate is clean and dried. A plastic microfluidic chip with modified surface characters is thus obtained.

In this invention, a direct write laser machining system is used to fabricate microfluidic channels on plastic substrate. To conduct the fabrication, the user designs pattern of the microfluidic system to be prepared in the substrate, using commercially available drawing-tools. The pattern is then converted into a format readable by the laser scriber. Light source of the laser scriber may be a CO₂ laser beam. The laser beam is directed to the plastic substrate to scribe trenches described by the pattern. Variable trench depth and width are fabricated by adjusting the laser power, beam motion speed, number of passes of laser and/or the beam spot size.

Although there are many ways to produce the pattern on the plastic substrate to form a microfluidic chip, several advantages are obtained by using the direct write laser machining in this invention. First of all, the overall development time for a microfluidic chip is largely reduced since a desired pattern can be generated and put to work in hours instead of weeks. Second, channels with varying aspect ratios can be easily fabricated in the same substrate. Complex microfluidic patterns can be fabricated without difficulties. Third, the large machining area allows production in large quantity. For example, in a plastic substrate of 31 cm×61 cm, 450 2 cm×2 cm patterns may be prepared at one time within an hour. Further extension in production flexibility and capacity can be easily achieved by employing more steps. Furthermore, when a direct laser is used, the trench cross section is consistent across the entire machining area. Of course, other laser scriber (such as laser scriber using a swivel mirror) or physical (such as casting) or chemical (such as chemical etching)—microfluidic channel forming technologies may be used in this invention to form the microfluidic channel pattern, as long as microfluidic channels with desired features may be produced in a plastic substrate.

Any plastic material may be used as the substrate to produce the microfluidic chip. Among the suited materials, PMMA (poly (methyl methacryolate)) substrate is recommended mainly because it is a non-porous solid and, therefore, contamination caused by biomolecule absorption is diminished. Besides, PMMA is inert in neutral aqueous solution and no hydrolysis will occur during the application.

There are currently two types of PMMA sheets available commercially. They are the cast PMMA and the extruded PMMA. The cast PMMA has higher softening temperature than that of the extruded PMMA. Although the roughness is reduced when extruded PMMA sheet is used, occasional bulges are still present. The cast PMMA sheet shows a rougher surface after machining. However, the cross section distortion at the rim is negligible.

Physical surface modification to smoothen the rugged surface after the laser machining is conducted by thermal annealing. The chip is then subject to a chemical surface modification process with the incorporation of versatile function groups. Surface of the chip is treated by surface hydroxyl group. Organosilanes are applied for further introduction of various groups, such as perfluoroalkyl (—CₙF₂ₙ₊₂), amino (—NH₂) or sulfhydryl (—SH). Surface modification agents as used in the embodiments of this invention includes: 1H, 1H, 2H, 2H-Perfluorodecyltriethoxysilane, 3-(Aminopropyl)trimethoxysilane and 3-Mercaptotrimethoxysilane. The modified surface can be used for the immobilization of biomolecules such as in the application of DNA and protein microarray. The introduction of other functional groups can also be performed similarly by the use of corresponding organosilanes.

The invented method does not require extensive solvent drying procedures. Straightforward chemical reduction of the ester group on the PMMA surface by lithium aluminum hydride (LAH) produces hydroxyl groups (—OH). The functional group is associates to widely used silanization chemistry. Hundreds of organosilanes bearing various organic functional groups are commercially available.

Although it is not intended to limit the scope of this invention, the chemical reaction systems of the chemical reduction action of LAH (a) and of the surface modification reaction systems of 1H, 1H, 2H, 2H-Perfluorodecyltriethoxysilane, 3-(Aminopropyl)trimethoxysilane and 3-Mercaptotrimethoxysilane (b) are shown, respectively, in Table I. Table I illustrates the chemical reaction systems of the chemical reduction action and the surface modification reaction of this invention.

The surface smoothness after thermal annealing is examined by scanning electron microscopy (SEM). The surface chemical modification is confirmed by attenuated total reflectance-Fourier transform infrared spectroscopy (ATR-FTR) and X-ray photoelectron spectroscopy (XPS). The whole procedure provides a very efficient micromachining platform to fabricate plastic microfluidic ship for both prototyping and mass production of biochips.

### EMBODIMENTS

Detailed description of the embodiments of this invention will be given below. It shall be understood that description of the embodiments is only for illustration purposes and shall not be used to limit the scope of the present invention.

A PMMA substrate is prepared. A commercial laser scriber is used to engrave the PMMA substrate. The CO₂ laser has wave length of 10.6 μm and its full power is 25 W. Data of a microfluidic pattern designed using commercially available design tools are provided to the laser scriber for direct machining on the PMMA substrate. To form a channel, the trench is sealed by thermally bonding the pattern substrate with a blank PMMA substrate at 120° C. for 30 minutes. Additional thermal annealing process at 170° C. for 30 minutes is performed to enhance the surface smoothness.

The PMMA sheet so prepared is treated with thermal annealing. The annealing is performed in ambient air with elevated temperature at 170° C. for 30 minutes.

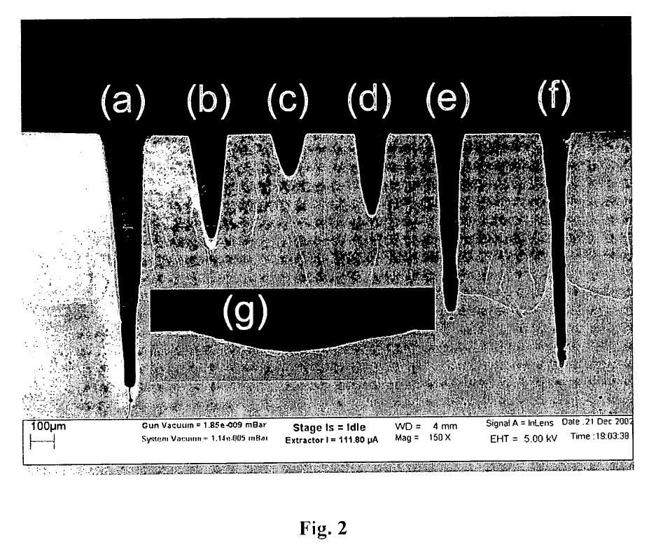

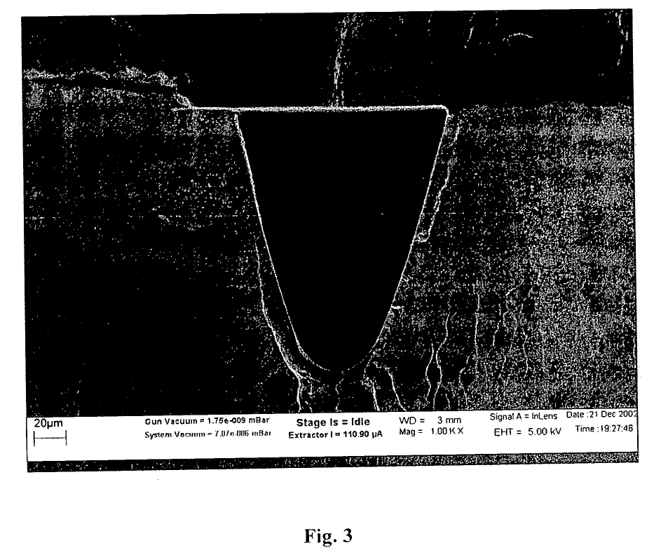

A field emission scanning electron microscope (SEM, Leo 1530, Leo, UK) is used to measure the cross section of the channel. FIG. 2 shows the cross-sectional view of trenches in cast PMMA carved with various later power and passes, after being thermally annealed. In FIG. 2, (a), (b) and (c) grooves are machined with laser power of 7.5 W, 3 W and 1.5 W, respectively. (d), (e) and (f) are grooves carved with 1.5 W laser power with 2, 5 and 10 passes, respectively. The inset (g) shows a wide-shallow groove carved with 1.5 W laser power and at speed of 36 mm/sec. The samples are subjected to thermal annealing before taking the pictures. FIG. 3 shows the SEM close-up of a sealed channel carved with 1.5 W laser power and at speed of 36 mm/sec., after being thermally annealed. These figures show the cross section remains unchanged after thermal annealing and bonding. In FIG. 3, the straight boundary between the two PMMA sheets indicates that the refill on the rim does not occur.

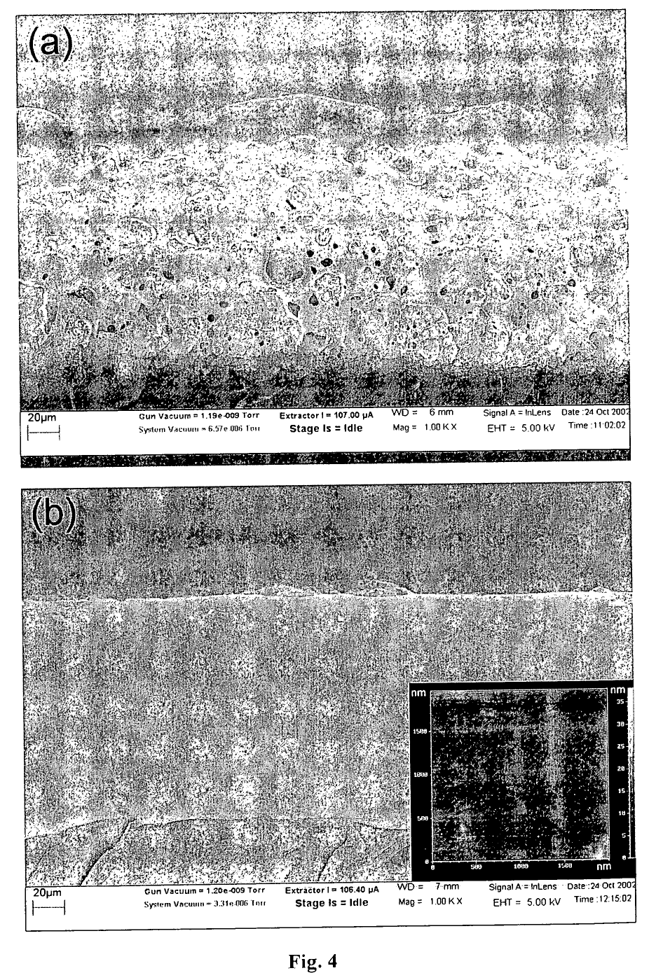

FIG. 4a shows an SEM picture of the rugged interior surface of the trench after laser machining and FIG. 4b shows the SEM picture of the same surface after thermal annealing. The AFM topography of the annealed surface is shown in the inset with full scale of 38 mm in the Z-axis. The viewing angle is perpendicular to the plain of the side wall. The roughness of the non-annealed surface is about 5-10 μm and decreases to 2.04±0.61 nm (rms) after the thermal annealing.

The PMMA sheet is later cut into chips of desired size, then sonicated in 2-propanol, rinsed with deionized water (resistivity 18.2 MΩ-cm, Millipore) and dried in a stream of dry nitrogen. The chips are then put in lithium aluminum hydride (LAH) solution in diethyl ether for 24 hours. 16 g of LAH in 100 ml in ether is used for the chemical reduction. The solution is constantly agitated during reaction. For the incorporating of organosilanes, the PMMA chips are first cleaned thoroughly by sonication in 2-propanol. The chips are then immersed in the solution of 1H, 1H, 2H, 2H-Perfluorodecyltriethoxysilane, 3-(Aminopropyl)trimethoxysilane and 3-Mercaptotrimethoxysilane, respectively, in 2-propanol (5% v/v) for 1 hour. The chips are then rinsed thoroughly with 2-propanol and then cured in a vacuum oven at 80° C. for 16 hours.

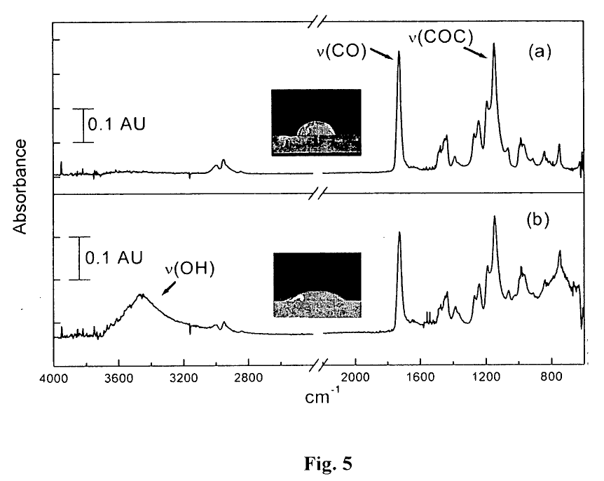

The attenuated total reflectance-Fourier transform infrared (ATR-FRIT) spectra of the chips are obtain using an IR-Plan Advantage Microscope (Spectra Tech., USA), attached to a spectrometer (Excalibur FTS-3000, Bio-Rad, USA). The substrate is subjected to hot baking at 80° C. for 24 hours to remove surface bound water. Each spectrum represents the average of 256 scans in 4 cm−1 resolution. FIG. 5a shows the ATR-FTIR spectra of the pristine PMMA surface and FIG. 5b shows the ATR-FTIR spectra of the LAH treated PMMA surface. The insets show the corresponding pictures form the measurement of contact angle.

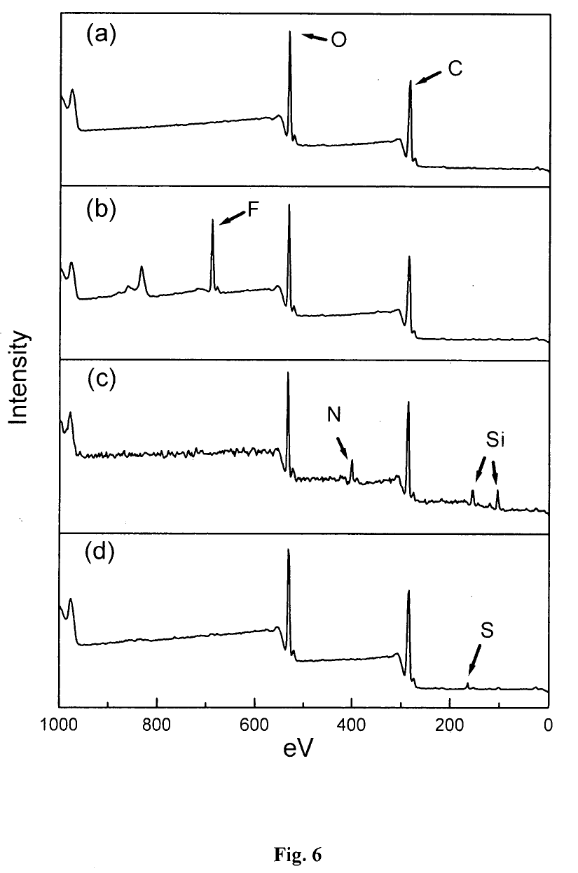

The X-ray photoelectron spectra of the chips are collected on a spectrometer (ESCALAB 250, VG Scientific, UK) using XR5 monochromatic X-ray gun with a power of 300 W. The spectrum is an average of three scans with 2 eV/step and with pass energy of 10 eV. The dwell time is 100 ms. The binding energy of carbon and oxygen in PMMA are 286.0 eV and 534.0 eV, respectively. FIGS. 6a-d show the XPS spectra of various organosilane modified surfaces according to the method of this invention. FIG. 6a shows the spectrum of the pristine PMMA after treatment with 3-(Aminopropyl)trimethoxysilane. FIGS. 6b, 6c and 6d show the spectra of the LAH treated PMMA substrate reacted with 1H, 1H, 2H, 2H-Perfluorodecyltriethoxysilane, 3-(Aminopropyl)trimethoxysilane and 3-Mercaptotrimethoxysilane, respectively.

In FIGS. 6a-d, characteristic energy corresponding to surface element is indicated. In FIG. 6a, no heteroatoms except oxygen present on PMMA surface when pristine PMMA is reacted with 3-(Aminopropyl)trimethoxysilane. The absence of heteroatoms in FIG. 6a indicates that no physical adsorption of the organosilane occurs on pristine PMMA surface. FIG. 6c is taken at a portion inside a trench. The result shows no apparent difference to the XPS spectra taken at portions of the flat surface. In addition, the immobilized silane is stable in the thermal condition used in bonding procedure since, as known, the elevated temperature (120° C.) used in glass silanization does not affect the stability of the silane.

The organosilane modification largely expands the application of the PMMA as a biochip substrate. The perfluoroalkylation modification can be used for surface passivation of PMMA to prevent adsorption. The aminoalkylation and mercaptoalkylation have been used for biomolecule immobilization.

Using the present invention, aspect ratios greater than 7 can be easily achieved. In FIG. 2, the aspect ratio is 1.2, 2.4, 5.2 and 7.0, with 1, 2, 5, and 10 passes, respectively. A higher aspect ratio is possible as long as the PMMA can sustain the structure form collapse.

As the present invention has been shown and described with reference to preferred embodiments thereof, those skilled in the art will recognize that the above and other changes may be made therein without departing form the spirit and scope of the invention.

1. Method for preparation and surface modification of plastic microfluidic chip, comprising:
prepare a plastic substrate is prepared;
form a pattern of trench with desired trench size and aspect ratio in said plastic substrate;
subject said substrate to physical surface modification;
treat surface of said substrate is treated with chemical reduction to produce hydroxyl groups (—OH); and
treat said substrate with a surface modification agent.

2. The method according to claim 1 wherein said plastic substrate is a PMMA substrate.

3. The method according to claim 1 wherein said plastic substrate is carved with a laser scriber to form said pattern.

4. The method according to claim 3 wherein said plastic substrate is carved with a direct write laser scriber to form said pattern.

5. The method according to claim 1 wherein said physical surface modification comprises thermal annealing treatment.

6. The method according to claim 1 wherein said reduction agent comprises lithium aluminum hydride (LAH).

7. The method according to claim 1 wherein said surface modification agent comprises at least one chemical selected from the group consisted of chemicals with functional groups of perfluoroalkyl (—CₙF₂ₙ₊₂), amino (—NH₂) or sulfhydryl (—SH).

8. The method according to claim 1 wherein said surface modification agent comprises fluorinated organosilanes.

9. The method according to claim 1 wherein said surface modification agent comprises aminated organosilanes.

10. The method according to claim 1 wherein said surface modification agent comprises thiolated organosilanes.

11. The method according to claim 1 wherein said surface modification agent comprises at least one chemical selected from the group consisted of 1H, 1H, 2H, 2H-Perfluorodecyltriethoxysilane, 3-(Aminopropyl)trimethoxysilane and 3-Mercaptotrimethoxysilane.

12. A plastic microfluidic chip prepared according to any one of claims 1-11.
